(19) United States
(12) Patent Application Publication
(10) Pub. No.: US 2023/0181430 A1
(43) Pub. Date: Jun. 15, 2023

(54) TOPICAL FORMULATIONS COMPRISING DUTASTERIDE FOR TREATING DERMATOLOGICAL DISORDERS INCLUDING MALE PATTERN BALDNESS

(71) Applicant: XYON Health Inc., Vancouver (CA)

(72) Inventors: Victor Hasson, Vancouver (CA); Mauro Castiglioni, Lentate sul Seveso (IT)

(73) Assignee: XYON Health Inc., Vancouver (CA)

(21) Appl. No.: 17/922,748

(22) PCT Filed: May 3, 2021
(86) PCT No.: PCT/US2021/030519

§ 371 Date: Nov. 1, 2022

Related U.S. Application Data
(60) Provisional application No. 63/019,335, filed on May 2, 2020.

(51) Int. Cl. A61K 8/14; A61K 8/893; A61K 8/899; A61K 8/55; A61Q 7/00

(52) U.S. Cl. CPC A61K 8/14; A61K 8/553; A61K 8/893; A61K 8/899; A61Q 7/00

(57) ABSTRACT

The present disclosure provides a liposomal topical formulation comprising dutasteride having barrier-forming siliconic components, methods for making the same, and methods for using the same in the treatment of dermatological diseases and conditions, including male pattern baldness.

### 1. BACKGROUND

Pattern hair loss in both men and women can be androgenic. It is generally believed that male sex hormones play a major role in the health of hair follicles. Hair follicles contain androgen receptors that bind with androgens: the resulting complexes could cause miniaturization of hair follicles and reduce nutrients being supplied to hair shafts. Although androgen receptors are present on scalp in general, they are at higher concentrations on balding scalp (e.g., in smaller follicles) than on non-balding scalp.

Anti-androgens can be useful for management of hair loss. For instance, finasteride is the active ingredient in an oral formulation (marketed under the brand name Propecia®) for treating male pattern baldness. Finasteride’s mechanism of action is inhibition of 5-α-reductase, the enzyme that converts testosterone into a more potent androgen, dihydrotestosterone (DHT). Finasteride can effectively lower DHT levels in scalp, which can arrest the progression of hair loss or promote new hair growth.

Although finasteride can be used to effectively treat hair loss in men, it is associated with a number of significant undesirable systemic side effects, which in turn can lead to poor patient compliance. For example, use of finasteride has been linked to low libido, erectile dysfunction, decreased arousal, and problems with orgasm. See, e.g., Irwig, M.S., Kolukula, S. J. Sex. Med. 8(6), 1747-53 (2011). Although suppression of serum DHT may be a general marker for these and other undesirable side effects, suppression of serum DHT may also be required in order to achieve the desired effect of treating hair loss. See FDA-approved label for Propecia®, Merck & Co, Inc., Whitehouse Station, NJ.

Another 5-α-reductase inhibitor, dutasteride, is the active ingredient in an oral formulation (marketed under the brand name Avodart®) for treating benign prostatic hyperplasia (BPH). Dutasteride, being a dual inhibitor of two isoforms of 5-α-reductase is even more effective at suppressing serum DHT levels upon oral administration. Although dutasteride and finasteride are chemically similar, dutasteride is more lipophilic (logP 6.8 versus 3.03), has higher plasma protein binding (99% versus 90%), and has a much high volume of distribution (300 to 500 L versus 44 to 96 L) than finasteride, which can alter pharmacological properties significantly. Dutasteride is approved for oral administration to treat male androgenetic alopecia in Japan at a dosage of 0.1 or 0.5 mg per day. Nonetheless, oral administration of dutasteride is also associated with an increased risk of undesirable side effects. See, e.g., Choi, G.S., et al. Ann. Dermatol. 28(4), 444-50 (2016).

Accordingly, a need exists in the art for a composition that can deliver active pharmaceutical ingredients (APIs) topically, such as dutasteride, to the skin, such as the scalp, in a dose effective to treat pattern hair loss while reducing systemic uptake of the same so as to reduce side effects.

### 2. SUMMARY

The present disclosure provides pharmaceutical formulations comprising dutasteride for topical dermatological use. In some embodiments, the formulations are particularly suited as a local depot for sustained and/or slow release of dutasteride as an active pharmaceutical ingredient (API). The topical formulations described herein are particularly suitable for treating pattern baldness, such as male-pattern baldness. The disclosed formulations provide highly localized delivery of dutasteride to the balding scalp for arresting, delaying, or reversing hair loss. The topical formulations described herein, in some instances, provide a “depot” effect by which dutasteride is largely retained in the outer layers of the skin, e.g., epidermis and part of the dermis, which are not as well-vascularized as the deeper parts of the skin, resulting in significantly lower serum concentrations of dutasteride as compared with oral administration of dutasteride. The resulting lower serum concentrations of dutasteride are correlated with reduced serum DHT suppression (i.e., higher serum DHT concentrations). Given the association of serum DHT suppression with a number of adverse effects (e.g., sexual dysfunction), these topical formulations offer a more favorable risk-benefit profile when compared to the corresponding oral formulations due to the resultant reduced systemic exposure.

One embodiment provides a liposomal topical formulation base comprising: a plurality of liposomes; and an aqueous gel matrix in which the plurality of liposomes are dispersed, wherein the aqueous gel matrix comprises a gelling agent, a water-soluble silicone compound, a film forming agent, and water. Examples of film forming agents include polyacrylamides, polyacrylates, polyvinylpyrrolidones, and co-polymers of these, and polysilsesquioxane compounds, preferably a poly(alkylsilsesquioxane) and most preferably poly(methylsilsesquinoxane). The base can be supplied to a user, e.g., a compounding pharmacy, for addition of dutasteride. In certain embodiments, the compounding of the base and dutasteride results in the entrapment of dutasteride within the liposomes of the base.

Thus, one embodiment provides a liposomal topical formulation comprising: a plurality of liposomes; an aqueous gel matrix in which the plurality of liposomes are dispersed, wherein the aqueous gel matrix comprises a gelling agent, a water-soluble silicone compound, a film forming agent, and water; and dutasteride, wherein dutasteride is entrapped within the liposomes. Examples of film forming agents include polyacryl amides, polyacrylates, polyvinylpyrrolidones, and co-polymers of these, and polysilsesquioxane compounds, preferably a poly(alkylsilsesquioxane) and most preferably poly(methylsilsesquinoxane).

One embodiment provides a liposomal topical formulation comprising: a plurality of liposomes, an aqueous gel matrix in which the plurality of liposomes are dispersed, wherein the aqueous gel matrix comprises a gelling agent, a water-soluble silicone compound, and water; and dutasteride, wherein dutasteride is entrapped within the liposomes. In a preferred embodiment, the aqueous gel matrix further comprises a film forming agent. Examples of film forming agents include polyacrylamides, polyacrylates, polyvinylpyrrolidones, and co-polymers of these, and polysilsesquioxane compounds, preferably a poly(alkylsilsesquioxane) and most preferably poly(methylsilsesquinoxane).

One embodiment provides a method for treating or preventing a dermatological disorder such as male pattern baldness, the method comprising administering a liposomal topical formulation according to the various embodiments disclosed herein.

Certain other embodiments provide compositions that have utility over a broad range of therapeutic applications, and may be used to treat diseases, such as androgenic alopecia (male pattern baldness), fungal infections, bacterial infections, acne, eczema, psoriasis, rosacea, vitiligo, and inflammation. Accordingly, another embodiment provides methods for treating or preventing a dermatological disease or condition, the method comprising administering to a patient in need of such a treatment a therapeutically effective amount of a composition disclosed herein.

### 3. BRIEF DESCRIPTION OF THE FIGURES

FIGS. 1A-E depict scalp hair growth in subjects before and following daily topical administration of a dutasteride formulation (2% w/w) according to an embodiment of this disclosure. More specifically, FIG. 1A depicts a 1st subject at 0 months (left) and after 3 months (right) of treatment. FIG. 1B depicts a 2nd subject at 0 months (left) and after 3 months (right) of treatment. FIG. 1C depicts a 3rd subject at 0 months (left) and after 3 months (right) of treatment. FIG. 1D depicts a 4th subject at 0 months (left) and after 3 months (right) of treatment. FIG. 1E depicts a 5th subject at 0 months (left) and after 3 months (right) of treatment.

FIGS. 2A and 2B depict scalp hair growth in subjects before and following weekly topical administration of a dutasteride formulation (2% w/w) according to an embodiment of this disclosure. More specifically, FIG. 2A depicts a 1st subject at 0 months (left) and after 2 months (right) of treatment. FIG. 2B depicts a 2nd subject at 0 months (left) and after 2 months (right) of treatment.

### 4. DETAILED DESCRIPTION

In the following disclosure, certain specific details are set forth in order to provide a thorough understanding of various embodiments. However, one skilled in the art will understand that the methods and uses described herein may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

### 4.1. Definitions

As used in the specification and appended claims, unless specified to the contrary, the following terms and abbreviations have the meaning indicated:

“Alkoxy” refers to a radical of the formula -ORₐ where Rₐ is an alkyl radical as defined below containing one to twelve carbon atoms.

“Alkyl” refers to a straight or branched hydrocarbon chain radical consisting solely of carbon and hydrogen atoms, containing no unsaturation, having from one to twelve carbon atoms, and which is attached to the rest of the molecule by a single bond. Preferably, the alkyl radial has one to eight carbon atoms, more preferably one to six carbon atoms. Examples of alkyl radicals include methyl, ethyl, n-propyl, 1-methylethyl (isopropyl), n-butyl, n-pentyl, 1,1-dimethyl ethyl (t-butyl), 3-methylhexyl, 2-methylhexyl, and the like.

“Antibiotic” or “antibiotic agent” refers to compounds or a combinations of compounds that are destructive to or inhibit the growth of microorganisms including bacteria, protozoa and/or microbes. As used herein, the term “antibiotic” includes antibacterial agents, antimicrobial agents, and the like. Examples of antibiotic agents include, but are not limited to, penicillins, cephalosporins, polymyxins, rifamycins, lipiarmycins, quinolones, sulfonamides, macrolides, lincosamides, tetracyclines, cyclic lipopeptides, glycylcyclines, oxazolidinones, and lipimycins.

“Antifungal” or “fungicide” refers to compounds or combinations of compounds that treat or prevent mycoses (i.e., kill or inhibit the growth of fungi). Antifungals include, but are not limited to, polyenes (e.g., amphotericin B, candicidin, filipin, hamycin, natamycin, nystatin, rimocidin), imidazoles (e.g., bifonazole, butoconazole, clotrimazole, econazole, fenticonazole, isoconazole, ketoconazole, luliconazole, miconazole, omoconazole, oxiconazoie, sertaconazole, sulconazole, tioconazole), triazoies (e.g., albaconazole, efinaconazole, epoxiconazole, fluconazole, isavuconazole, itraconazole, posaconazole, propiconazole, ravuconazole, terconazole, voriconazole), thiazoies (e.g., abafungin), allylamines (e.g., amoroifin, butenafine, naftifine, terbinafme), echinocandins (e.g., amdulafungin, caspofungin, micafungin), aurones, benzoic acid, cielopirox, flucytosine, griseofulvin, haloprogin, tolnaftate, undecylenic acid, crystal violet, and Balsam of Peru.

“Aryl” refers to a hydrocarbon ring system radical comprising hydrogen, 6 to 18 carbon atoms and at least one aromatic ring. For purposes of this disclosure, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene.

“Film forming agents” refers to organic or inorganic polymer or oligomers, or organic-inorganic copolymers that are capable of forming a cohesive film or mesh.

“Subject” refers to an animal, such as a mammal, for example a human.

As used herein, “n” is typically an integer up to 100.

### 4.2. Embodiments

Disclosed herein are liposomal topical formulations capable of highly-localized, slow release of dutasteride as an active pharmaceutical ingredient (API). Advantageously, due to a local depot effect and barrier function provided by the siliconic components of certain embodiments of the formulations disclosed herein, dutasteride penetrates through the stratum corneum of the epidermis and is largely confined to the epidermis and dermis. As a result, while a therapeutically effective local (skin) concentration of dutasteride can be maintained; systemic exposure of dutasteride is reduced. As a result, in certain instances, the liposomal topical formulations provide a therapeutic effect while reducing or eliminating side effects associated with systemic exposure of dutasteride, such as erectile dysfunction, sexual dysfunction, and the like.

In various embodiments, the liposomal topical formulations disclosed herein may be loaded with dutasteride, at suitable strengths; or may act as a base into which dutasteride could be added.

One embodiment thus provides a liposomal topical formulation comprising dutasteride, a plurality of liposomes entrapping dutasteride, an aqueous gel matrix in which the plurality of liposomes are dispersed, wherein the aqueous gel matrix comprises a gelling agent, a water-soluble silicone compound, a film forming agent such as a polysilsesquioxane compound, and water.

Another embodiment provides a liposomal topical base formulation comprising a plurality of liposomes; an aqueous gel matrix in which the plurality of liposomes are dispersed, wherein the aqueous gel matrix comprises a gelling agent, a water-soluble silicone compound, a film forming agent such as a polysilsesquioxane compound, and water.

One or more optional additives may be present in the topical formulations disclosed herein. These additives include, for example, one or more solvents, co-solvents, humectants, viscosity modifiers, antioxidants, stabilizers, penetration enhancers, and the like, including combinations thereof.

In certain embodiments, the topical formulations disclosed herein provide for controlled release of dutasteride. In some embodiments, the control release of dutasteride provides low system exposure. For instance, in some embodiments, the topical formulations provide serum dutasteride levels from 0.01 ng/mL to 5 ng/mL. In some embodiments, the topical formulations provide serum dutasteride levels from 0.1 ng/mL to 2 ng/mL, such as from 0.5 ng/mL to 1.5 ng/mL or from 1 ng/mL to 2 ng/mL. In certain embodiments, the topical formulations provide serum dutasteride levels from 0.5 ng/mL to 2 ng/mL, such as from 0.5 ng/mL to 1.5 ng/mL or 1 ng/mL to 2 ng/mL, when the formulation is applied to the scalp of a subject daily for 28 days. In some embodiments, the steady state serum dutasteride is from 0.5 ng/mL to 2 ng/mL, such as from 0.5 ng/mL to 1.5 ng/mL or 1 ng/mL to 2 ng/mL.

In certain embodiments, the topical formulations disclosed herein provide for decreased suppression of serum DHT levels. In certain embodiments, the topical formulations comprising dutasteride disclosed herein provide for decreased suppression of serum DHT levels compared to the same compositions but comprising finasteride. For instance, in some embodiments, the topical dutasteride formulations decrease suppression of serum DHT levels at least 75% less than the corresponding finasteride formulations, such as at least 65%, 50%, 35%, 25%, 20%, 15%, 10%, or 5% less than the corresponding finasteride formulations, particularly at least 75%, or 25% less. In certain embodiments, the topical dutasteride formulations decrease suppression of serum DHT levels from 5%, 8%, or 10% to 15%, 18%, 20%, or 25% less than the corresponding finasteride formulations, such as from 5-25% less, 8-25% less, 10-25% less, 5-20% less, 8-20% less, 10-20% less, etc. In some embodiments, the topical dutasteride formulations decrease suppression of serum DHT levels from 10%, 15%, 20%, or 25% to 30%, 40%, 50%, 65%, or 75% less than the corresponding finasteride formulations, such as from 10-75% less, 15-75% less, 20-75% less, 25-75% less, 10-50% less, 15-50% less, 20-50% less, 25-50% less, etc.

In some embodiments, the topical formulations provide serum DHT suppression from 1% to 35%. In some embodiments, the topical formulations provide serum DHT suppression from 1% to 25%, such as from 1% to 15% or from 5% to 20%. In certain embodiments, the topical formulations provide serum DHT suppression from 1% to 25%, such as from 1% to 15% or from 5% to 20% when the formulation is applied to the scalp of a subject daily for 28 days. In some embodiments, the topical formulations provide steady state serum DHT suppression from 1% to 25%, such as from 1% to 15% or from 5% to 20%. In some embodiments, the topical formulations disclosed herein provide no statistically significant serum DHT suppression (e.g., do not reduce serum DHT).

The topical formulations, whether or not loaded with dutasteride or another API, have the consistency and spreadability of a gel or lotion. The topical formulations may also be in the forms of cream, spray, foam, serum, and the like.

These and other optional additives are described in further detail below.

### 4.2.1. Liposomes

Liposomes are small vesicles comprising amphiphilic lipids arranged in bilayers. Liposomes may contain several concentric lipid bilayers separated by aqueous channels (multilamellar vesicles or MLVs), or alternatively, they may contain a single membrane bilayer (unilamellar vesicles), which may be small unilamellar vesicles (SUVs) or large unilamellar vesicles (LUVs).

The vesicle-forming amphiphilic lipids are preferably ones having two hydrocarbon chains, typically acyl chains, and a polar head group. There are a variety of synthetic vesicle-forming lipids and naturally-occurring vesicle-forming lipids, including the phospholipids, such as phosphatidylcholine, phosphatidyl ethanolamine, phosphatidic acid, phosphatidylinositol, and sphingomyelin, where the two hydrocarbon chains are typically between 12-40, or more typically 14-22 carbon atoms in length. The hydrocarbon chains may contain varying degrees of unsaturation (i.e., 0 to up to 6 double bonds). The two hydrocarbon chains (or fatty chains) are covalently linked to a polar head group, which typically comprises an ionized moiety such as phosphate or ammonium. The above-described lipids and phospholipids can be obtained commercially or prepared according to known methods in the art. Other suitable lipids include glycolipids and sterols, such as cholesterol.

In a preferred embodiment, the vesicle-forming lipid is lecithin, an amphiphilic compound typically derived from animal and plant tissues (e.g., egg yolks or soya beans). In general, lecithin includes a diglyceride of two fatty acids such as stearic, palmitic, and oleic acids. The diglyceride is coupled (via the third hydroxyl group) to a phosphoric acid, which incorporates a choline moiety. The lipids of the lecithin group are also commonly called phosphatidylcholines. Lecithin can be hydrogenated in a controlled manner to yield hydrogenated lecithin. In some embodiments, the lecithin is a hydrogenated lecithin, for example, Lecinol S-10 (Nikkol Group, Nikko Chemical Co., Ltd.).

In certain embodiments, in an aqueous medium, the amphiphilic lipid molecules spontaneously arrange into vesicles having a bilayer membrane defining an aqueous interior compartment. Composed of two lipid monolayers, the bilayer membrane has a hydrophobic region wherein the tails of the two lipid monolayers orient toward the center of the bilayer and a hydrophilic region wherein the heads of the lipid monolayer orients toward the aqueous interior of the vesicles and the aqueous medium in which the liposomes are dispersed.

The amount of the vesicle-forming lipid in an aqueous medium must reach a critical concentration to form stable liposomes. Typically, lecithin may be present in an amount of 0.1-5% (w/w) by the total weight of the topical formulation. More typically, the amount may be in the range of 0.1 -4%, 0.1-3%, 0.1-2%, 0.1-1%, and 0.5-1% and the like.

### 4.2.2. Active Pharmaceutical Ingredient (API)

Liposomes are capable of solubilizing both water-soluble and lipid-soluble compounds, making them effective carriers of APIs. For a given API, depending on its solubility in water or lipid, it may be trapped in the aqueous interior compartment, the lipid bilayer, or both according to a partition coefficient. Polar APIs (e.g., salts) are hydrophilic and are largely trapped in the aqueous compartment; whereas nonpolar APIs are largely trapped in the lipid bilayer.

Liposomes are dynamic structures. The entrapped API, whether within the aqueous interior or the lipid bilayer or both, can be released in a slow or controlled manner. In some embodiments, the liposomal topical formulation entraps all, substantially all, or a portion of the API in the liposomes. For example, in certain embodiments, at least 75, 80, 85, 90, 95, or 98% of the total API, e.g., dutasteride, in the formulation is entrapped within the liposomes. In a particular embodiment, at least 90% of the total API, e.g., dutasteride, is entrapped within the liposomes.

In a preferred embodiment, the API is dutasteride. In various alternative embodiments, suitable APIs are topically active agents that treat or reduce the symptoms of various dermatological disorders. Examples of such alternative APIs include, without limitation, an 5-α-reductase inhibitor other than dutasteride (such as finasteride), an antifungal agent, an antibiotic, an anti-hypertensive vasodilator, a steroid, an anti-acne agent, a topical anti-inflammatory agent, and combinations thereof. While many of the topical formulations described herein are discussed in the context of dutasteride as API, it is understood that finasteride can also be used in the alternative.

Dutasteride, a synthetic 4-azasteroid compound, is a 5-α-reductase inhibitor. 5-α-reductase has three isoforms, all involved in converting testosterone to dihydrotestosterone (DHT). 4-Azasteroid compounds are lipophilic and can be efficiently trapped in the lipid bilayers of liposomes of the topical formulations disclosed herein and released in a highly localized manner. In some embodiments, the high lipophilicity of dutasteride (i.e., experimental logP 6.8; calculated logP 5.45-5.79) permits slow and highly localized release into the scalp of a subject useful for treating male-pattern baldness. In certain embodiments, dutasteride is released from the topical formulations disclosed herein into the scalp of a subject and decreases scalp DHT concentrations while reducing systemic exposure of dutasteride. Limiting the systemic exposure of dutasteride reduces serum DHT suppression and potential undesirable side effects.

For avoidance of doubt, all formulations, including liposomal topical formulations, described herein may include dutasteride as an API in an effective amount therein. Dutasteride (structure shown below) inhibits all three 5-α-reductase isoforms and is capable of suppressing up to 99% of DHT production after sustained oral administration:

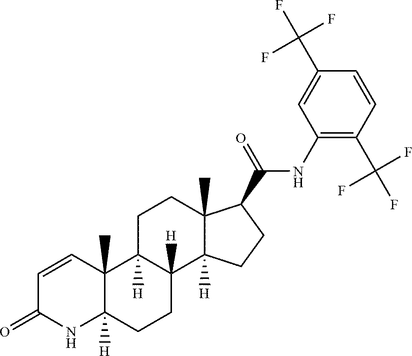

Finasteride (structure shown below) selectively inhibits Type II and Type III 5-α-reductase, typically reducing serum DHT levels by about 65-70% after sustained oral administration. Because finasteride does not inhibit Type I 5-α-reductase, it does not fully suppress DHT production.

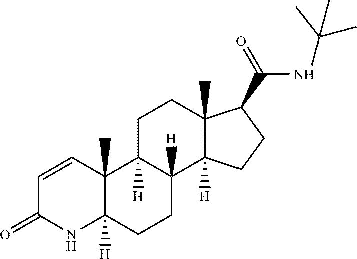

In certain embodiments, dutasteride is the only API in the formulations described herein, including liposomal topical formulations. In other embodiments, dutasteride is included as an API in the formulation in combination with one or more additional APIs as described herein, such as one or more additional steroids, such as 5-α-reductase inhibitors, such as finasteride. In some embodiments, the formulation comprises a combination of dutasteride and finasteride as APIs in an effective amount therein. In some of such embodiments, dutasteride and finasteride are the only APIs in the formulation.

Other embodiments provide non-steroidal 5-α-reductase inhibitors. These compounds are not technically steroids; however, they also have fused or non-fused cyclic structures and are considered structural analogs of certain azasteroids. Suitable compounds include benzo[c]quinolizinones (e.g., bexlostende), benzo[f]quinolonones, piperidones, and the like. In further embodiments, the API is saw palmetto extract, melatonin, or both saw palmetto extract and melatonin.

In yet other specific embodiments, the APIs may be dermatologically active agents that target infections, acne, pigmentation, premature aging, and the like. Without limitation, examples of such APIs include psoralens, macrolides, retinoids, azole-based antifungal agents, allylamines, morpholino-based antifungal agents, selenium-based antifungal agents, hydroquinones, potassium channel openers, tetracyclines, monoxidil, and combinations thereof.

It is noted that suitable APIs for the present disclosure are not limited to the above specific examples. Rather, the liposomal topical formulation disclosed herein can be loaded with any API suitable for transdermal delivery. The API may be loaded at a suitable amount depending on its efficacy, at strengths suitable for the treatment or prophylaxis of a particular disorder. A 5-α-reductase inhibitor may be loaded at 0.5-5% (w/w) of the total weight of the topical formulation. More preferably the amount is in the range of 0.5-4%, 0.5-3%, 1-4%, 1-3%, 2-3%, 2-4%, 2-5%, 2.5-4%, 4-5% and the like. In certain embodiments, dutasteride is present at 0.5%, 1%, 1.5%, 2%, 2.5%, 3%, 3.5%, 4%, 4.5%, or 5% (w/w) of the total weight of the topical formulation, or any ranges created by using these amounts as endpoints. In preferred embodiments, dutasteride is present at 0.5-5%, 1-4%, or 1-3%, such as 1%, 1.5%, 2%, 2.5%, or 3% (w/w), particularly 2% (w/w) of the total weight of the topical formulation. In some embodiments, dutasteride is present from 1%, 1.5%, or 2% to 3%, 3.5%, 4%, 4.5%, or 5% (w/w) of the total weight of the topical formulation. In other embodiments, dutasteride is present from 0.5%, 1%, or 1.5% to 2.5%, 3%, 3.5%, 4%, 4.5%, or 5% (w/w) of the total weight of the topical formulation. In certain embodiments, dutasteride is present from 0.5%, 1%, 1.5%, 2%, or 2.5% to 3.5%, 4%, 4.5%, or 5% (w/w) of the total weight of the topical formulation.

### 4.2.3. Gelling Agent

A gelling agent is a hydrophilic polymer that is insoluble in water, but can absorb water and swell into up to 1000 times its original volume. The gelling agent, having absorbed water and having swollen into a gel, provides a matrix in which the liposomes can be uniformly dispersed.

Suitable gelling agents include, for example, acacia, alginic acid, bentonite, Carbopols (now known as carbomers), cellulose-based polymers, gelatin, poloxamers (Pluronics), polyvinyl alcohol, sodium alginate, tragacanth, and xanthan gum.

In a preferred embodiment, the gelling agent is a carbomer, which refers to a class of polymers of acrylic acid or acrylate (esters of acrylic acid) crosslinked by for example, divinyl glycol and polyalkenyl ethers. Carbomers readily absorb water without dissolving in water. The crosslinked structure allows the polymer to swell and form a gel-like consistency. Examples of polyacrylate polymers include, without limitation, polyacrylonitrile, polyacrylic acid, and alkyl acrylate crosspolymers.

Polyacrylate polymers also include, but are not limited to, polyacrylic acid, polymethacrylic acid, polymethyl methacrylate, poly butylacrylate, poly 2-ethylhexyl acrylate, and poly(C₁₀-C₃₀ alkyl acrylate) crosspolymers. In certain embodiments, the acrylate polymer comprises a C₁₀-C₃₀ alkyl acrylate crosspolymer, for example, Carbopol Ultrez® 21 (Lubrizol Advanced Materials, Inc.).

In other embodiments, one or more cellulose-based polymers, such as carboxymethyl cellulose, ethylcellulose, hydroxyethyl cellulose, hydroxypropyl cellulose, and methylcellulose, are used as gelling agents. Gelling agents may be used at concentrations of 0.5% to 10% by weight (such as from 1%, 2%, or 3% to 7%, 8% or 9% by weight, or any ranges created by using these amounts as endpoints), depending on the agent and the target viscosity of the formulation. For carbomers (e.g., Carbopol Ultrez® 21), the amount by weight of the total topical formulation may be 0.1-5%. In some embodiments, the amount is in the range of 0.5-4%, 0.1-4%, 0.1-3%, 0.5-2%, 0.5-1% and the like. In certain embodiments, the carbomer may be present at 0.5%, 0.6%, 0.7%, 0.8%, 0.9%, 1%, 1.5%, or 2% (w/w) of the total weight of the topical formulation, or any ranges created by using these amounts as endpoints. In preferred embodiments, the carbomer may be present at 0.5%, 0.7%, 0.8%, 0.9%, or 1% (w/w), particularly 0.8% (w/w), of the total weight of the topical formulation.

### 4.2.4. Water-Soluble Silicone Compounds

Silicone, also known to as polysiloxane, is a class of organosilicon polymer having a plurality of silicon-carbon bonds and siloxane linkages (—Si—O—Si—) in the polymer backbone. Polysiloxane is typically a linear polymer but may be modified to contain branches or pendants of other chemical moieties or polymers. The most common silicone is poly(dimethylsiloxane), or PDMS or dimethione, which has the following structure:

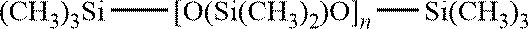

wherein n is an integer and the number of n determines the molecular weight, viscosity, density of the polydimethylsiloxane.

Unmodified PDMS is highly hydrophobic and not compatible with an aqueous-based formulation. However, PDMS (or other polysiloxanes) can be modified to contain hydrophilic pendants or capping groups. Such hydrophilic groups include, for example, polyols such as polyethylene glycol (PEG) and polypropylene glycol (PPG). Examples of modified, water-soluble silicones include PEG-modified silicones or PPG-modified silicones.

The PEG or PPG moieties may be appended to any one or more of the repeating units by replacing one or more methyl groups or coupling to a modified methyl group having reactive groups such as hydroxyl. The PEG or PPG moieties may also replace the one or both of the end methyl group of PDMS. As the number of the ethylene oxide or propylene oxide units appended to the PDMS chain increases, the hydrophilicity increases and the modified PDMS becomes more and more water-soluble. Hydrophilicity can be measured by the hydrophile-lipophile balance (HLB) number.

As used herein, the molecular weight of the silicone portion of the water-soluble silicone compound is less than 10,000, and more preferably, less than 8,000, or less than 6,000. In various embodiments, n is in the range of 20-100, more preferably, 20-80, or still more preferably, 30-60.

In an embodiment, at least 6 ethylene oxide or propylene oxide units are present for each molecule of PDMS for the PEG or PPG-modified silicone to be appreciably water-soluble. More preferably, at least 8 ethylene oxide or propylene oxide units are present for each molecule of PDMS. In various embodiments, the water-soluble silicone is represented by PEG-X silicone or PPG-X silicone, wherein x is an integer between 6 and 40, wherein x represents the number of ethylene oxide units. More preferably, x is 8-20, and still more preferably, x is 8-15.

In a preferred embodiment, the water-soluble silicone compound is PEG-8 dimethicone, for example Silwax® WS-4 (Siltech Corporation, Toronto, Ontario).

In other embodiments, the water-soluble silicone compound is Bis-PEG-18 methyl ether dimethylpolysiloxane, wherein the two capping groups of the polysiloxane chain are PEG-18 (18 units of ethylene oxide).

The water-soluble silicone compound is used at concentrations of 0.5-20% or more, preferably 5-20% (w/w) of the total weight of the topical formulation. In various embodiments, the amount of the water-soluble silicone compound is in the range of 5-15%, or 5-10% or 8-15% or 8-10% and the like. In preferred embodiments, the PEG-8 dimethicone may be present at 8%, 9%, 10%, 11%, or 12% (w/w), or any ranges created by using these amounts as endpoints, particularly 12% (w/w), of the total weight of the topical formulation.

### 4.2.5. Film Forming Agents

In some embodiments, the film-forming agents are polymers or co-polymers that contain a hydrophilic moiety in their repeating units. Examples of film forming agents include polyacrylamides, polyacrylates, polyvinylpyrrolidones, and co-polymers of these. In preferred embodiments, the film forming agents are polysilsesquioxane compounds.

A polysilsesquioxane compound is a branched siloxane polymer having a chemical formula [RSiO_{3/2}]ₙ, wherein n is an integer greater than zero and R is at each occurrence independently H, alkyl, aryl, hydroxy or alkoxy.

Polysilsesquioxane compounds generally adopt a cage-like or ladder-like structure with Si—O—Si linkages. In a preferred embodiment, the polysilsesquioxane compound is a poly(alkylsilsesquioxane). Still more preferably, the polysilsesquioxane compound is poly(methylsilsesquinoxane) (i.e., R is methyl), which is commercially available under the brand name Gransil PSQ® (Grand Industries, Inc., Elmwood Park, NJ). Polysilsesquioxane compounds are especially preferred film-forming polymers. When applied topically (including on the scalp), a polysilsesquioxane compound, alone or together with the water-soluble silicone, is capable of forming a barrier, thereby slowing down the penetration and diffusion of the API. It is believed that the polysilsesquioxane compound forms a 3D mesh structure when applied to the skin. The mesh structure serves to entrap the liposomes in a mask-like covering of the scalp. In certain embodiments, the API (e.g., dutasteride) is released in a controlled fashion into the skin, where it is thereafter retained.

The polysilsesquioxane compound is present at concentrations of 1-20% (w/w) of the total weight of the topical formulation. In various embodiments, the amount of the water-soluble silicone is in the range of 1-10%, or 5-10% or 1-5% or 10-20% and the like, of the total weight of the topical formulation. In a preferred embodiment, the polysilsesquioxane compound is Gransil PSQ®. In some embodiments, the polysilsesquioxane compound (e.g., Gransil PSQ®) is present in the range of 1-10%, such as 2%, 3%, 4%, 5%, 6%, 7%, 8% or 9%, preferably 4-6%, or 5% of the total weight of the topical formulation. In some embodiments, the polysilsesquioxane compound (e.g., Gransil PSQ®) is present in at least 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% (w/w) of the total weight of the topical formulation. Still more preferably, the Gransil PSQ® is present in at least 3%, at least 4%, or at least 5% (w/w) of the total weight of the topical formulation. In a preferred embodiment, the Gransil PSQ® is present in at least 3% or at least 5% (w/w) of the total weight of the topical formulation.

### 4.2.6. Optional Additives

Solvents and co-solvents suitable for the topical formulation include, without limitation, one or more alcohols and isosorbide.

Alcoholic solvents can be any solvent having at least one hydroxyl group. Alcoholic solvents can be miscible with both water and organic substances (such as PEG-silicone and polysilsesquioxane). The alcoholic solvent may be, without limitation, propanediol, phenoxyethanol, aminomethyl propanol, ethanol, iso-propanol, tert-butanol, and the like.

The optional additive may further include isosorbide, which is a versatile solvent that is compatible with both water and organic solvents. Isosorbide refers to a compound having the following structure:

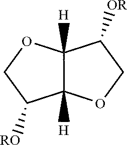

wherein R at each occurrence, independently H or C₁-C₆ alkyl. When at each occurrence R is methyl, the above compound is dimethyl disosorbide (or DMI), which is commercially available under the brand name Gransolve® DMI (Grant Industries, Elmwood Park, NX).

The optional additive may further include a humectant, which helps to reduce moisture loss after the topical application is applied. An example of a suitable class of humectant is glyceryl derivatives, which refers to compounds derived from glycerol (1,2,3-propanetriol), wherein one or more of the hydrogens of the hydroxyl groups of glycerol are replaced by a straight or branched alkyl, alkenyl, or alkynyl chain. Glyceryl derivatives include mono-, di- and tri-esters of glycerin, for example, glyceryl caprylate (caprylyl glycol), ethylhexylglycerin, and mixtures thereof. In some embodiments, the glyceryl derivative is caprylyl glycol, for example Neofect® 403 (IMCD N.V., Rotterdam, Netherlands).

In other embodiments, the composition optionally includes one or more preservatives to inhibit microbial activity. Suitable preservatives include mercury-containing substances such as merfen and thiomersal; stabilized chlorine dioxide: and quaternary ammonium compounds such as benzalkonium chloride, cetyltrimethylammonium bromide and cetylpyridinium chloride.

Still other embodiments of the composition include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkyethers and alkylphenyl ethers, e.g., octoxynol 10, octoxynol 40.

Still other embodiments of the composition include one or more antioxidants to enhance chemical stability where required. Suitable antioxidants include, by way of example only, butylated hydroxytoluene, butylated hydroxyanisole, Vitamin E, ascorbic acid and sodium metabisulfite.

### 4.2.7. Method of Making the Topical Formulation

The topical formulation of the present disclosure can be made sequentially by first forming the liposomes and then the gel matrix. Vesicle-forming lipids are mixed with water while stirring, whereby the lipids form into liposomes under the shear force of the mixing. To this mixture, a gelling agent, water-soluble silicone compound and, if desired, a polysilsesquioxane compound can be added under stirring, until a homogeneous and uniform formulation is formed.

Additional optional additives can be added at any point of the process, preferably after the liposome formation.

As discussed herein, a liposomal topical base formulation may be formed without the API. The base formulation takes the form and consistency of a gel and thereafter can be loaded with one or more API(s).

Alternatively, the API is loaded during the formation of the liposome or after the formation of the liposome but before the formation of the gel matrix (i.e., before adding a gelling agent).

The siliconic components, including water-soluble silicone compound and, if present, a polysilsesquioxane compound may be added to the liposomes (with or without API). These siliconic components are film-forming compounds and contribute to the depot effect of the API release from the topical formulation.

### 4.2.8. Use of the Topical Formulation

The topical formulation disclosed herein may be applied to the skin, including scalp, of a subject. The methods described herein can be useful for both human therapeutics and veterinary applications. In some embodiments, the subject is a mammal, and in some embodiments, the subject is human.

As discussed herein, due to the depot effect, in certain embodiments, the API may be released slowly, or in a controlled fashion, and in a highly localized manner. In some embodiments, formulations prepared according to the present disclosure permit reduced steady state plasma concentrations (e.g., 1.5- to 5.5-fold less) than known topical formulations with no siliconic components. In some embodiments, the flux and amount of API, e.g., dutasteride, permeating the skin may be greatly reduced using formulations prepared according to embodiments of the present disclosure.

In some embodiments, the topical use of the API, such as dutasteride, leads to faster results then the oral use of the API for treating a dermatological disorder, such as male pattern baldness. In some embodiments, the amount of API, such as dutasteride, used for treating a dermatological disorder is described in the formulations herein.

In some embodiments, the formulation is administered in multiple doses. In some embodiments, dosing is once, twice, three times, four times, five times, six times, or more than six times per day, particularly once a day. In other embodiments, dosing is once a month, once every two weeks, once a week, or once every other day. In preferred embodiments, the formulation comprising finasteride or dutasteride is administered once every other day, once every three days, once every four days, once every five days, once every six days, or twice, three times, four times, five times or six times a week. In some embodiments, the formulation is administered once every other day. In some embodiments, the formulation is administered once every three days. In some embodiments, the formulation is administered once every four days. In some embodiments, the formulation is administered once every five days. In some embodiments, the formulation is administered once every six days. In some embodiments, the formulation is administered twice a week (e.g., on Monday and Thursday). In some embodiments, the formulation is administered three times a week (e.g., on Monday Wednesday, and Friday). In some embodiments, the formulation is administered four times a week (e.g., on Monday, Wednesday, Friday, and Saturday). In some embodiments, the formulation is administered five times a week (e.g., on Monday, Tuesday, Wednesday, Friday, and Saturday). In some embodiments, the formulation is administered six times a week. In preferred embodiments, the formulation is administered once a week or at longer intervals. In some embodiments, the formulation comprising finasteride or dutasteride is administered once a week, once every 8 days, once every 9 days, once every 10 days, once every 11 days, once every 12 days, once every 13 days, or once every two weeks. Administration at weekly or longer intervals of the formulation comprising dutasteride is especially preferred. Administration less often than daily (e.g., administration once a week) surprisingly is therapeutically efficacious, particularly for the formulation comprising dutasteride, while reducing the risk of exposing a household member (e.g., a spouse or child) of the treated patient to an active ingredient, such as finasteride or dutasteride. In yet another embodiment, the administration continues for more than 6, 10, 14, 28 days, two months, six months, or one year. In some cases, continuous dosing is achieved and maintained as long as necessary to achieve the desired effect, such as hair growth. For example, in one embodiment, a composition of any one of the foregoing embodiments is administered once per day for 3 weeks.

Administration of the composition may continue as long as necessary. In some embodiments, a composition is administered for more than 1, 2, 3, 4, 5, 6, 7, 14, or 28 days. In some embodiments, a composition is administered chronically on an ongoing basis, e.g., for the treatment or prophylaxis of chronic conditions.

Dermatological conditions or disorders that may be addressed or treated by the topical formulations include, without limitation, androgenic alopecia (male pattern baldness), infection (a bacterial or fungal infection), acne, eczema, psoriasis, rosacea, vitiligo, inflammation, pain, itch, and the like. More specifically, such diseases and symptoms may include by way of example and not limitation, a cutaneous condition, skin cancer, mycosis, dermatitis, a blister, scabies, a skin infection (e.g., fungal, bacterial, or other microbial), allergic reaction, erythema, skin ulcer, contact dermatitis, seborrheic dermatitis, skin infection, acne, atopic dermatitis, melanoma, warts, vitiligo, psoriasis, skin rash, hives, pustule, herpes simplex, ringworm, autoimmune disease, xeroderma, lupus erythematosus, impetigo, keratosis, basal-cell carcinoma, squamous cell skin cancer, nodules, rosacea, hyperpigmentation, burns (e.g., first degree, second degree, third degree, sunburns), cysts, lichen planus, skin puncture or cut (i.e., wounds), shingles, bullous pemphigoid, ichthyosis, molluscum contagiosum, athlete’s foot, alopecia areata, folliculitis, cellulitis, pemphigus, pityriasis, and candidiasis.

### 5. EXAMPLES

Pharmacokinetic studies were conducted to determine serum DHT and dutasteride concentrations of the described dutasteride compositions delivered topically and their effect was studied in the treatment of subjects with male patterned baldness.

### Example 1: Preparation of a 2.5% W/W Finasteride Topical Composition

A sanitized turbo-emulsifier was charged with water, followed by Lecinol S-10. The resultant mixture was stirred at high speed for 10 minutes at ambient temperature. While maintaining stirring, the mixture was warmed to 70° C. and stirred for an additional 60 minutes at 70° C. Carbopol Ultrez® 21 was added and the resultant mixture was stirred at medium speed at 70° C. for 10 minutes and allowed to stand at a temperature of 70° C. for 30 minutes, ensuring the acrylate polymer was solvated. To the mixture was added a solution of API (finasteride) in 1,3-propane diol and the resultant mixture was stirred at high speed for 10 minutes at 70° C. While maintaining stirring, the mixture was allowed to cool to 30° C. and Neofect® 403, phenoxyethanol, Silwax® WS, and dimethyl isosorbide were added sequentially. To the resultant homogeneous mixture was added 2-amino-2-methylpropan-l-ol followed by stirring at high speed for 10 minutes. Thereafter, Gransil PSQ® was added to the mixture, followed by 10 minutes of high speed stirring to afford a homogeneous gel. The final concentration of each respective component is shown in Table 1 below:

TABLE 1

Component concentrations for a 2.5% w/w finasteride topical composition

| Component (Commercial Name) | Concentration (% w/w) | INCI Name |
|---|---|---|
| Purified water | 59.9 | Aqua |
| Lecinol S-10 | 0.5 | Hydrogenated Lecithin |
| Carbopol Ultrez® 21 | 0.8 | Acrylates/C 10-30 Alkyl Acrylate Crosspolymer |
| Finasteride | 2.5 | - |
| 1,3-propane diol | 10.0 | Propanediol |
| Neofect® 403 | 0.8 | Caprylyl Glycol/Glyceryl Caprylate/Ethylhexylglycerin |
| Phenoxyethanol | 0.2 | Phenoxyethanol |
| Silwax® WS | 10.0 | PEG-8 Dimethicone |
| Gransolve® DMI | 10.0 | Dimethyl Isosorbide |
| AMP | 0.3 | Aminomethyl propanol |
| Gransil PSQ® | 5.0 | Poly(methylsilsesquioxane) |

### Example 2: Preparation of 1% and 2% W/W Dutasteride Topical Compositions

A sanitized turbo-emulsifier was charged with water, followed by Lecinol S-10. The resultant mixture was stirred at high speed for 10 minutes at ambient temperature. While maintaining stirring, the mixture was warmed to 70° C. and stirred for an additional 60 minutes at 70° C. Carbopol Ultrez® 21 was added and the resultant mixture was stirred at medium speed at 70° C. for 10 minutes and allowed to stand at a temperature of 70° C. for 30 minutes, ensuring the acrylate polymer was solvated. To the mixture was added a solution of API (dutasteride) in 1,3-propane diol and the resultant mixture was stirred at high speed for 10 minutes at 70° C. While maintaining stirring, the mixture was allowed to cool to 30° C. and Neofect® 403, phenoxyethanol, Silwax® WS, and dimethyl isosorbide were added sequentially. To the resultant homogeneous mixture was added 2-amino-2-methylpropan-1-ol followed by stirring at high speed for 10 minutes. Thereafter, Gransil PSQ® was added to the mixture, followed by 10 minutes of high speed stirring to afford a homogeneous gel. The final concentration of each respective component is shown in Table 2 below:

TABLE 2

Component concentrations for 1% and 2% w/w dutasteride topical compositions

| Component (Commercial Name) | Concentration (% w/w) in 2% composition | Concentration (% w/w) in 1% composition | INCI Name |
|---|---|---|---|
| Purified water | 60.4 | 61.4 | Aqua |
| Lecinol S-10 | 0.5 | 0.5 | Hydrogenated Lecithin |
| Carbopol Ultrez® 21 | 0.8 | 0.8 | Acrylates/C 10-30 Alkyl Acrylate Crosspolymer |
| Dutasteride | 2 | 1 | - |
| 1,3-propane diol | 10.0 | 10.0 | Propanediol |
| Neofect® 403 | 0.8 | 0.8 | Caprylyl Glycol/Glyceryl Caprylate/Ethylhexylglycerin |
| Phenoxyethanol | 0.2 | 0.2 | Phenoxyethanol |
| Silwax® WS | 10.0 | 10.0 | PEG-8 Dimethicone |
| Gransolve® DMI | 10.0 | 10.0 | Dimethyl Isosorbide |
| AMP | 0.3 | 0.3 | Aminomethyl propanol |
| Gransil PSQ® | 5.0 | 5.0 | Poly(methylsilsesquioxane) |

### Example 3: Preparation of Gel Base

A sanitized turbo-emulsifier was charged with water, followed by Lecinol S-10. The resultant mixture was stirred at high speed for 10 minutes at ambient temperature. While maintaining stirring, the mixture was warmed to 70° C. and stirred for an additional 60 minutes at 70° C. Carbopol Ultrez® 21 was added and the resultant mixture was stirred at medium speed at 70° C. for 10 minutes and allowed to stand at a temperature of 70° C. for 30 minutes, to achieve solvation of the acrylate polymer. While maintaining stirring, the mixture was allowed to cool to 30° C. and Nefect® 403, phenoxyethanol, Silwax® WS, and dimethyl isosorbide were added sequentially. To the resultant homogeneous mixture was added 2-amino-2-methylpropan-1-ol followed by stirring at high speed for 10 minutes. Gransil PSQ® was added to the mixture, followed by 10 minutes of high speed stirring to afford a homogeneous gel. The final concentration of each respective component is shown in Table 3 below:

TABLE 3

Component concentrations for a representative gel base

| Component (Commercial Name) | Concentration (% w/w) | INCI Name |
|---|---|---|
| Purified water | 72.4 | Aqua |
| Lecinol S-10 | 0.5 | Hydrogenated Lecithin |
| Carbopol Ultrez® 21 | 0.8 | Acrylates/C 10-30 Alkyl Acrylate Crosspolymer |
| Neofect® 403 | 0.8 | Caprylyl Glycol/Glyceryl Caprylate/Ethylhexylglycerin |
| Phenoxyethanol | 0.2 | Phenoxyethanol |
| Silwax® WS | 10.0 | PEG-8 Dimethicone |
| Gransolve® DMI | 10.0 | Dimethyl Isosorbide |
| AMP | 0.3 | Aminomethyl propanol |
| Gransil PSQ® | 5.0 | Poly(methylsilsesquioxane) |

### Example 4: Preparation of a 2.5% W/W Finasteride Topical Composition via Compounding

A mixture of API (finasteride, 2.5 g) and the liposomal gel base of Example 3 (2.5 g) was levigated (i.e., milled together) at ambient temperature in an electronic mortar and pestle (e.g., Unguator E/S, Galenova, Inc., Saint-Hyacinthe, QC, Canada) for 5 minutes. Additional liposomal gel base (95 g) was added portion wise over 10 minutes and the levigation was continued until a visually-homogeneous mixture is obtained.

### Example 5: Preparation of 1% and 2% W/W Dutasteride Topical Compositions via Compounding

A mixture of API (dutasteride, 1 g and 2 g in the two compositions, respectively) and the liposomal gel base of Example 3 (2.5 g) was levigated (i.e., milled together) at ambient temperature in an electronic mortar and pestle (e.g., Unguator E/S, Galenova, Inc., Saint-Hyacinthe, QC, Canada) for 5 minutes. Additional liposomal gel base to a total of 100 g was added portion wise over 10 minutes and the levigation was continued until a visually-homogeneous mixture is obtained.

### Example 6: In Vitro Skin Permeation Assessment of Finasteride

A skin permeation study was used to determine the permeation profile of finasteride through human skin (epidermis) in vitro. Three different formulations were prepared, (1) a formulation prepared according to Example 1 (“Formulation 1”), (2) a carbomer-based gel without siliconic components or liposomes (“carb gel”), and (3) a liposome gel without siliconic components (“lipo gel”), each with a finasteride concentration of 2.5% w/w. Profiles for three different formulations through epidermis samples showed dramatically different permeation through epidermis as well as a calculated flux for Formulation 1 after 24 hours that was well below those of the other formulations tested. It can be observed that Formulation 1 showed the lowest permeation profile and a calculated flux that was greatly reduced relative to the other formulations tested.

In order to compare the in vitro and in vivo data, an equation was used to calculate the steady-state plasma concentration of finasteride estimated on the basis of in vitro permeation data, the equation is given below:

$${\text{C}}_{\text{SS}} \text{=} \left(\text{A*J}\right) / \text{CL}$$

Css is the steady state plasma concentration for finasteride; A is the skin area available for diffusion; J is the in vitro permeation rate (flux; µg/cm²h); and CL is the systemic clearance after oral administration (165 mL/minute for finasteride). The calculated Css value for each formulation was compared to the maximum concentration at steady state (Cmax) obtained after daily oral administration of 1 mg finasteride capsules.

The application area of the gel was assumed to be 200 cm² and the clearance value of 165 mL/minute was used to estimate the plasma steady state concentration (Css) from the in vitro experiments conducted with human skin from the same donor. These data are presented in Table 4 below.

TABLE 4

In vitro permeation and retention data for finasteride and human skin*

| | Permeated Amount After 24 hours (µg/cm²) | Estimated In vitro Permeated Amount 24 hrs (µg/cm²) | Flux (µg/cm²) | C₅₅ (ng/mL) | Retained Amount After 24 hours (µg/cm²) | Estimated In vivo Permeated Amount 24 hrs (µg/cm²) |
|---|---|---|---|---|---|---|
| Formul. 1 | 0.66 ± 0.24 | 0.016 | 0.02 ± 0.00 | 0.49 | 0.41 ± 0.58 | 0.081 |
| Carb Gel | 1.55 ± 0.06 | 0.034 | 0.04 ± 0.01 | 0.74 | 0.34 ± 0.21 | 0.066 |
| Lipo Gel | 4.11 ± 1.50 | 0.241 | 0.13 ± 0.01 | 2.69 | 0.49 ± 0.39 | 0.095 |

* Data reported as mean ± standard deviation

It was observed that when finasteride was administered to human skin in a formulation prepared according to embodiments of the present disclosure, the resulting steady state plasma concentration was between about 1.5- to 5.5-fold less than when administered according to other known topical formulations. It was also observed that the flux and amount of finasteride that permeated the skin was greatly reduced for formulations prepared according to embodiments of the present disclosure. As evidenced by the data in Table 10, these studies show the desirable “drug depot” qualities exhibited by the topical formulations prepared according to embodiments of the present invention.

### Example 7: In Vitro Skin Permeation Assessment of Dutasteride

To investigate the impact of the siliconic components in dutasteride-loaded formulations, in vitro tests were conducted to measure the permeation through and retention into skin epidermis of a 1% dutasteride-loaded silicone gel formulation made according to the process of Example 2. As a comparison, a gel spray of dutasteride without silicone (PEG-8 Dimethicone or Poly(methylsilsesquioxane)) was also prepared. An original Franz-type diffusion cell system was modified to accommodate widened vertical columns and removal of the bowl shape. The diffusion area was 0.636 cm² with a receiver capacity of approximately 3.0 mL. The receiver volume for each cell was individually calibrated.

### Skin Preparation

Human epidermis samples were prepared according to standard protocols. The samples were obtained from abdominal skin of two 30-50 year old Eurasiatic donors. The samples were used as a membrane between the two chambers in the Franz cells. After about 6-8 hours, fat cells were carefully removed from the full thickness skin. The skin sections were cut into 2.5 x 2.5 cm squares, sealed in aluminum foil and frozen at -20° C. Prior to preparation, the samples were thawed to room temperature, immersed in 60° C. water, and the epidermis was gently separated from the remaining tissue with forceps and allowed to dry.

### Skin Integrity

Before being mounted on the Franz cell, each epidermal sheet was visually inspected to avoid any possible defects. Additionally, the electrical resistance of isolated epidermis was measured to ensure the integrity of the barrier membrane. Epidermis samples with a resistance above 18 kΩ/cm³ were used for experiments.

### Skin Permeation

The upper and lower parts of the Franz cell were sealed with paraffin film and fastened together by a clamp. The membrane was positioned to act as a seal between the donor and receptor compartments. The skin was carefully mounted on the lower half of the Franz cell with the dermis facing downward and the stratum corneum in contact with the sample formulations. At the beginning of the experiment, the semisolid formulation was applied to the skin as donor phase (approximately 10 mg/0.636 cm) using an excavated silicone cylinder. The receiver compartments were filled with a saline solution, which had been filtered through a 0.2 µm membrane and sonicated under vacuum to remove air. Samples were prepared such that no air bubbles were present between the receptor medium and the dermis in the receptor compartment.

The prepared Franz cells were stirred continuously using a magnetic stir bar at a temperature of 37° C. At time points of 1, 3, 5, 7, and 24 hours, 0.2 ml samples were taken from the receiver compartment and replaced with fresh medium. Sink conditions were maintained throughout the experiments. Three replicates per test preparation per donor were performed. Samples from the receiver compartment were tested using gas chromatography to determine concentrations of compounds that permeated through the epidermis. Following the 24 hour permeation experiment described above, the epidermis samples were recovered, and any residual formulation was removed from the surface of the epidermis, followed by a 10 mL methanol wash to remove any additional residual formulation. The epidermis samples were cut into small pieces and collected in tubes containing 5 mL of methanol. Samples were sonicated for 30 minutes and left to stand. After standing for 24 hours, the supernatant was centrifuged at 3000 rpm for 10 minutes at 23° C. and analyzed by HPLC.

### Analysis of Dutasteride Concentration

Dutasteride concentration was determined using LC/MS-MS according to the following parameters:
Instrument: Acquity TQD (Waters) with ESI and triple quadrupole detector
Column: Poroshell 120 (Agilent) SB-C8 2.7 µm; 2.1 100 mm
Mobile Phase: Phase A (5 mM buffer, 0.1% formic acid / Phase B (acetonitrile)
Chromatographic Method: 20% Phase A/80% Phase B - isocratic for 7 minutes
Injection Volume: 10 µL
Column Temperature: 40° C.

Multiple Reaction Monitoring (MCM) was used for fragmentation at 529.4 > 95.27. Standards were prepared at concentrations of 10.5 and 1 µg/mL in methanol. Samples from the receiver compartment were injected without dilution.

### Results

In the receiver compartment, dutasteride was never detected, indicating that it did not permeate through human epidermis (skin) when subjected to the experimental conditions.

The retained amounts of dutasteride in the human epidermis samples after 24 hours using the two different formulations were not statistically different. The results are summarized in Table 5 below.

TABLE 5

In vitro permeation and retention data for dutasteride and human skin (n = 5)*

| | Permeated Amount After 24 hrs (µg/cm²) | Flux (µg/cm²) | Retained Amount After 24 hrs (µg/cm²) | % retained |
|---|---|---|---|---|
| Siliconic Formulation | Not detected | Not detected | 1.65 ± 0.58 | 0.81 ± 0.28 |
| Spray Formulation | Not detected | Not detected | 1.00 ± 0.45 | 0.49 ± 0.22 |

* Data reported as mean ± standard deviation

### Example 8: Finasteride Pharmacokinetic Studies

A pharmacokinetic (PK) and pharmacodynamic (PD) clinical study was performed to ascertain whether the topical liposomal formulation of Example 4 (i.e., a 2.5% w/w finasteride topical liposomal composition) leads to modest systemic exposure of finasteride in human subjects, and to determine whether daily application of the topical liposomal formulation of Example 4 to the scalp for three weeks leads to an attenuation of the reduction of plasma dihydrotestosterone (DHT) observed with conventional finasteride therapy.

### Bioanalysis

Ultra-high performance liquid chromatography/tandem mass spectrometry (UPLC-MS/MS) assays for the quantitation of finasteride and DHT in human plasma were developed and qualified. The assay for finasteride employed a stable isotope-labeled (deuterated) internal standard and was qualified over a linear range of 0.05 ng/mL (lower limit of quantitation) to 50 ng/mL (coefficient of determination = 0.9985) using an 8-point calibration curve. The signal-to-noise ratio at the lower limit of quantitation was > 10. Quality control (QC) standards were prepared using finasteride in blank human plasma at nominal concentrations of 0.2 ng/mL (low QC), 1 ng/mL (mid QC) and 40 ng/mL (high QC). The quality control standards were analyzed in quadruplicate and the concentration of finasteride interpolated from the linear calibration curve using peak area ratio methodology versus the internal standard. Each replicate calculated concentration was within ±13% of the nominal concentration for each of the low, mid and high QC standards. Blank samples were also injected at regular intervals in order to confirm the absence of analyte carry-over. The assay was therefore considered qualified for the accurate determination of finasteride in human plasma.

The assay for DHT employed a stable isotope-labeled (deuterated) internal standard and was qualified over a linear range of 50 ng/mL (lower limit of quantitation) to 10,000 pg/mL (coefficient of determination = 0.9988) using an 8-point calibration curve. The signal-to-noise ratio at the lower limit of quantitation was > 10. Quality control (QC) standards were prepared using DHT in blank human plasma at nominal concentrations of 50 pg/mL from the linear calibration curve using peak area ratio methodology versus the internal standard. Each replicate calculated concentration was within ±14% of the nominal concentration for each of the low, mid and high QC standards. Blank samples were also injected at regular intervals in order to confirm the absence of analyte carry-over. The assay was therefore considered qualified for the accurate determination of DHT in human plasma.

### Study Design

In a controlled study, six male subjects not previously treated with finasteride, and one male subject previously treated with finasteride but who had discontinued finasteride therapy for the preceding seven days, were administered a pretreatment blood draw to establish baseline levels of finasteride and DHT. No finasteride was detected in the previously untreated subjects; the previously treated subject had a baseline finasteride level of 0.098 ng/mL where the lower limit of detection was 0.05 ng/mL. The subjects were provided with the formulation of Example 4 and were instructed to apply it daily (as a thin layer) to the scalp for 20 consecutive days. The subjects were then instructed to return to the clinic on the day of their final dose (i.e., Day 21). After a pre-dose blood sample was taken, the subjects were administered their final (i.e., 21^{st}) dose. Blood samples were taken 1, 2, 4, 8 and 24 hours post-dose. The blood samples were processed to plasma and stored at -80° C. prior to UPLC/MS- MS analysis.

### Assay Performance and Quality Control

A pharmacokinetic (PK) assay was developed to detect finasteride and dihydrotestosterone (DHT) concentrations in human serum, respectively. Assay performance was within standard acceptance criteria (i.e., linearity of calibration curve, recovery of standards, bias of QC samples across operating range, etc). It was noted that Test Subject 1 had a pre-test finasteride serum concentration of 0.098 ng/mL (98 pg/mL), which was ascribed to inadequate pre-study wash-out. All other test subjects had finasteride concentrations below the lower limit of quantitation (i.e., < 50 pg/mL) of the assay, which was expected for the pre-test samples.

### Pharmacokinetic Analysis of 2.5% W/W Finasteride Topical Composition

After dosing once per day for 3 weeks with the 2.5% w/w finasteride topical liposomal composition of Example 4, plasma levels were tested to determine the finasteride serum concentration. Results indicated that finasteride serum concentrations were consistent over the 24 hour PK monitoring interval (i.e., between about 4.31 and 7.37 ng/mL) and showed no dramatic increase (i.e., “spike”) post-dose. In contrast, oral formulations of finasteride typically show a dramatic increase in serum finasteride concentration following dosage. The stable serum concentration in the topical treatment indicates a slow and steady egress of finasteride from the skin into the plasma. These data reflect the “depot” or “anchor” effect of the composition when a 2.5% w/w finasteride topical liposomal composition is administered. That is, the concentration of finasteride on the skin remains highly localized and the therapeutic agent (i.e., finasteride) is released slowly while the finasteride serum concentration does not show a dramatic or substantial increase. Total serum finasteride was measured (e.g., total bound plus total unbound). The results of the time course study are shown below in Table 6.

TABLE 6

Mean finasteride serum concentration over 24 hours at steady state

| Time Point (hrs) | Concentration ±σ (ng/mL) |
|---|---|
| 0 | 0.431 ± 0.282 |
| 1 | 0.397 ± 0.268 |
| 2 | 0.474 ± 0.275 |
| 4 | 0.495 ± 0.276 |
| 8 | 0.684 ± 0.346 |
| 24 | 0.737 ± 0.472 |

σ = standard deviation

The highest individual plasma concentration level detected was 1.390 ng/mL.

### Pharmacodynamic Analysis of 2.5% W/W Finasteride Topical Composition

After 3 weeks of treatment as described above, a modest reduction in the concentration of dihydrotestosterone (DHT) in serum of 31% ± 21% (range: 0-53%) was observed. By way of comparison, typical reduction in DHT serum concentration for oral formulations range from 60 to 70%. That is, the present compositions show an approximately 2-fold less reduction of DHT serum concentration when compared with oral finasteride therapy. The results were obtained by comparing the pre-test DHT serum concentration to the DHT serum concentration at T₀ of the last treatment (i.e., week 3). The data are shown below in Table 7.

TABLE 7

DHT serum concentrations pre-test and T₀ at week 3

| Test Subject | Pre-Test DHT Concentration (ng/mL) | Week 3 T₀ DHT Concentration (ng/mL) | Relative Decrease (%) |
|---|---|---|---|
| 1 | 0.214 | 0.214 | 0 |
| 2 | 0.320 | 0.169 | 47 |
| 3 | 0.391 | 0.249 | 36 |
| 4 | 0.665 | 0.325 | 51 |
| 5 | 0.602 | 0.545 | 9 |
| 6 | 0.608 | 0.287 | 53 |
| 7 | 0.287 | 0.225 | 22 |
| Mean | 0.441 | 0.287 | 31 |
| Standard Deviation | 0.180 | 0.124 | 21 |
| Median | 0.391 | 0.249 | 36 |

The DHT suppression data in Table 7 show a statistically-significant decrease in the mean concentration at Week 3 vs. pre-test (p = 0.0217, paired 2-sided Student’s t-test).

In addition, the time course mean DHT serum concentration was remarkably consistent over the 24 hour monitoring interval. The data show no sharp increase or drop in DHT serum concentration, which indicates slow, sustained release of finasteride. The results of the time course DHT serum concentration PD study are shown in Table 8.

TABLE 8

Mean DHT serum concentrations over 24 hours

| Time Point (hrs) | Concentration ±σ (ng/mL) |
|---|---|
| 0 | 0.287 ± 0.124 |
| 1 | 0.259 ± 0.137 |
| 2 | 0.298 ± 0.137 |
| 4 | 0.270 ± 0.116 |
| 8 | 0.246 ± 0.119 |
| 24 | 0.281 ± 0.130 |

σ = standard deviation

### Conclusions

The data demonstrate that the delivery of finasteride by the liposomal formulation of finasteride is largely restricted to the top layers of the skin. While slow, sustained release of finasteride into systemic circulation was observed, the plasma finasteride levels were generally very modest (in no cases exceeding 1.4 ng/mL). The mean reduction in plasma DHT by finasteride was far less pronounced with the composition of the liposomal formulation of finasteride (31%) than is seen with oral finasteride therapy (60-70%). Therefore, the data shows that the liposomal formulation of finasteride delivers finasteride to the skin with reduced systemic exposure and causes less reduction in systemic DHT levels than occurs with oral finasteride therapy.

### Example 9: Dutasteride Pharmacokinetic Studies

A pharmacokinetic (PK) clinical study was performed to ascertain whether the topical liposomal formulation of Example 5 (i.e., a 2% w/w dutasteride topical liposomal composition) leads to modest systemic exposure of dutasteride in human subjects, and to determine whether daily application of the topical liposomal formulation of Example 5 to the scalp for four weeks leads to an attenuation of the reduction of plasma dihydrotestosterone (DHT).

### Study Design

Subjects were selected (n = 10) that had not used oral or topical dutasteride at the time of enrollment or for at least 1 month prior to enrollment.
In the office, pre-treatment blood draws established baseline serum DHT concentration (i.e., [DHT]): first draw between 8:00-10:00 a.m. (T = 0), followed by draws at T = 1 h and T = 4 h to allow each subject to act as his own control for [DHT].
Starting immediately after final pre-treatment blood draw, treatment included one-a-day dosing (qd) with topical liposomal silicone dutasteride formulation for 4 weeks (28 days) to allow steady-state to be reached.
After 4 weeks, last dose was given in office between 8:00-10:00 a.m. and blood was drawn pre-dose (i.e., trough) and at 1 h, 2 h, 4 h, 8 h and 24 h post-dose for analysis of serum dutasteride concentration (i.e., [DUT]) and serum [DHT]. Rigorous exercise was avoided during the 2 days prior to these blood draws.

### Exclusion Criteria

Use of any concomitant medications which are in turn clinically significant inducers or inhibitors of CYP3A4;
Use of anabolic steroids, anti-androgen therapies or other androgen modulators in the past 6 months;
Use of any form of testosterone (e.g., topical, IM) in the past 6 months;
Unable/unwilling to refrain from consuming foods/herbs known to be clinically significant inducers or inhibitors of CYP3A4 (grapefruit juice, pomegranate juice, St. John’s wort);
Presence (or emergence during the course of the study) of scalp lesions or other significant skin barrier compromise at or near site of application;
Use of other topical medications on the scalp;
Donation or transfusion of plasma or blood within 30 days prior to drug administration. No planned donation or transfusion throughout the study, or for 30 days after, is allowed except for emergency reasons;
Any current infection or infection within 14 days before study drug administration;
Undergoing or have undergone treatment with another investigational drug (including antisense oligonucleotides), biologic agent or device within 4 weeks of screening;
History of pituitary or hypothalamic disease or radiation exposure;
History of depression, prostate cancer or castration; and
Unable/unwilling to abstain from sexual activity on the morning of any blood draws Results

Serum dutasteride and serum DHT PK data was collected in 9 subjects with data for T = 0 h and T = 4 h on Day 1, as well as for T = 1, 2, 4, 8 and 24 h on Day 28. Dutasteride data was also collected on Day 1 in a single subject.

### Serum Dutasteride

Mean serum dutasteride levels on Day 28 were modest (means by time point in the 1.2-1.5 ng/mL range). The Day 28 time course was essentially flat, consistent with the depot effects of the formulation in combination with being at expected steady state by Day 28. All Day 28 samples were within the range of 0.07-4.76 ng/mL. Total serum dutasteride was measured (e.g., total bound plus total unbound). Serum dutasteride concentrations in the PK study are summarized in Table 9.

TABLE 9

Serum Dutasteride Concentrations (ng/mL)

| Subject | Day 1 -0 h | Day 1 -4 h | Day 28 -1 h | Day 28 -2 h | Day 28 -4 h | Day 28 -8 h | Day 28 -24 h |
|---|---|---|---|---|---|---|---|
| 1 | < LLOQ | < LLOQ | 0.53 | 0.53 | 0.50 | 1.13 | 1.44 |
| 2 | < LLOQ | < LLOQ | 0.91 | 1.35 | 0.96 | 0.98 | 1.29 |
| 3 | < LLOQ | < LLOQ | ND | ND | ND | ND | ND |
| 4 | < LLOQ | < LLOQ | 1.24 | 1.95 | 1.18 | 0.74 | 1.34 |
| 5 | < LLOQ | < LLOQ | 1.02 | 1.05 | 1.39 | 1.31 | 1.95 |
| 6 | < LLOQ | 0.02 | 2.06 | 2.69 | 2.24 | 3.70 | 3.57 |
| 7 | < LLOQ | 0.04 | 4.76 | 3.99 | 2.71 | 0.84 | 0.79 |
| 8 | < LLOQ | < LLOQ | 0.72 | 0.76 | 0.66 | 0.24 | 0.12 |
| 9 | < LLOQ | < LLOQ | 0.26 | 0.12 | 0.07 | 1.57 | 1.26 |
| 10 | 0.52 | 0.52 | 1.37 | 1.42 | 1.51 | 0.09 | 0.08 |
| Mean | 0.052 | 0.058 | 1.43 | 1.54 | 1.25 | 1.18 | 1.32 |
| Range | 0 - 0.52 | 0 - 0.52 | 0.26 -4.76 | 0.12 -3.99 | 0.07 -2.71 | 0.09 -3.70 | 0.08 -3.57 |

< LLOQ = beneath the method’s lower limit of quantitation (0.02 ng/mL) - treated as “0” for mean calculation; ND = no data (no sample available) - excluded from mean calculation. Serum DHT

Serum DHT concentrations in the PK study are summarized in Table 10.

TABLE 10

Serum DHT Concentrations (ng/mL)

| Subject | Day 1 -0 h | Day 1 -4 h | Day 28 -1 h | Day 28 -2 h | Day 28 -4 h | Day 28 -8 h | Day 28 -24 h |
|---|---|---|---|---|---|---|---|
| 1 | 1.132 | 1.363 | 1.124 | 0.920 | 1.001 | 0.875 | 0.981 |
| 2 | 1.301 | 1.164 | 1.011 | 1.019 | 1.019 | 1.049 | 1.263 |
| 4 | 1.335 | 1.404 | 1.104 | 0.896 | 1.128 | 0.671 | 0.781 |
| 5 | 1.211 | 0.815 | 0.758 | 0.929 | 0.937 | 0.488 | 0.967 |
| 6 | 0.317 | 0.208 | 0.679 | 0.595 | 0.550 | 0.486 | 0.693 |
| 7 | 0.721 | 0.933 | 0.543 | 0.529 | 0.588 | 0.433 | 0.667 |
| 8 | 0.517 | 0.836 | 0.658 | 0.526 | 0.434 | 0.814 | 0.911 |
| 9 | 1.032 | 0.975 | 0.811 | 0.850 | 0.911 | 0.978 | 1.001 |
| 10 | 0.928 | 0.628 | 0.823 | 0.991 | 1.058 | 0.884 | 1.118 |
| Mean | 0.944 | 0.925 | 0.835 | 0.806 | 0.848 | 0.742 | 0.931 |
| Range | 0.317 -1.335 | 0.208 -1.404 | 0.543 -1.124 | 0.526 -1.019 | 0.434 -1.128 | 0.433 -1.049 | 0.667 -1.263 |

When the DHT levels at the 4 h-post dose time point on Day 28 vs. Day 1 were compared, the median DHT showed a decrease of 12% while the mean DHT showed an increase of 11%. Relative DHT data are summarized in Table 11.

TABLE 11

Relative Serum DHT Concentrations (4 h post-dose Day 28 vs. 4 h post-dose Day 1, ng/mL)

| Subject | Day 1 - 4 h | Day 28 -4 h | Relative DHT Suppression |
|---|---|---|---|
| 1 | 1.363 | 1.001 | 27% |
| 2 | 1.164 | 1.019 | 12% |
| 4 | 1.404 | 1.128 | 20% |
| 5 | 0.815 | 0.937 | -15% |
| 6 | 0.208 | 0.550 | -165% |
| 7 | 0.933 | 0.588 | 37% |
| 8 | 0.836 | 0.434 | 48% |
| 9 | 0.975 | 0.911 | 6% |
| 10 | 0.628 | 1.058 | -68% |
| | | Mean | -11% |
| | | Median | 12% |

The DHT data in Table 11 shows no statistically-significant changes at 4 h post dose for Day 28 vs. on Day 1 (p = 0.4795, paired 2-sided Student’s t-test).

### Example 10: Clinical Topical Dutasteride Study

A clinical study was performed to assess the efficacy associated with the dutasteride formulation described in Example 5. Patients in the study were positively identified as having androgenic alopecia (male pattern baldness). The degree of baldness was subsequently assessed according to the Norwood Hamilton Scale. Individuals with more advanced loss on this scale were selected, as changes is hair loss density and new hair growth would be more easily assessed in these cases. Exclusion criteria included the use of dutasteride or any other hair loss treatment and/or hormonal therapy within the prior six months.

Patients were instructed to apply the 2% w/w dutasteride topical liposomal topical gel to the areas of the scalp affected by pattern baldness massaging the gel into the scalp. The patients were instructed to apply the gel once daily after a morning shower, after having towel-dried the hair. Patients were instructed to not use hair-styling products during the course of the study.

All patients were informed of the known side-effects of oral dutasteride. They were instructed to contact the physician conducting the study should they experience any of these side-effects. In addition, they were instructed to report any other symptoms experienced, including local issues at the site of gel application.

Follow-up assessments were initiated by patients once they had observed significant changes in their hair growth. Further follow-up assessments were performed at 6 monthly intervals, or time of visit of the study physician, depending on the patient’s location. These assessments were documented using global photography of the scalp, focusing on areas affected by pattern baldness.

### Results

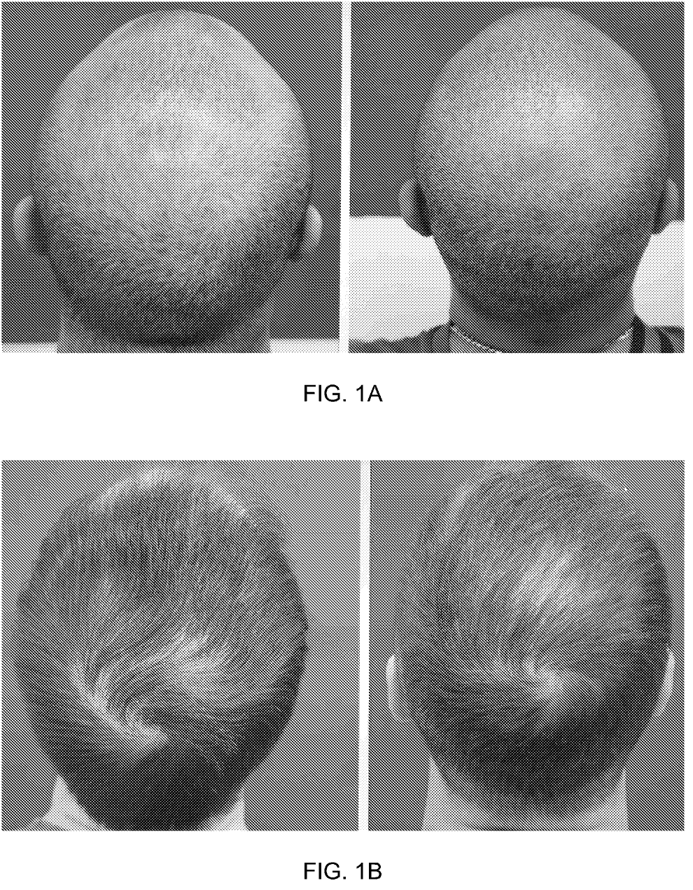

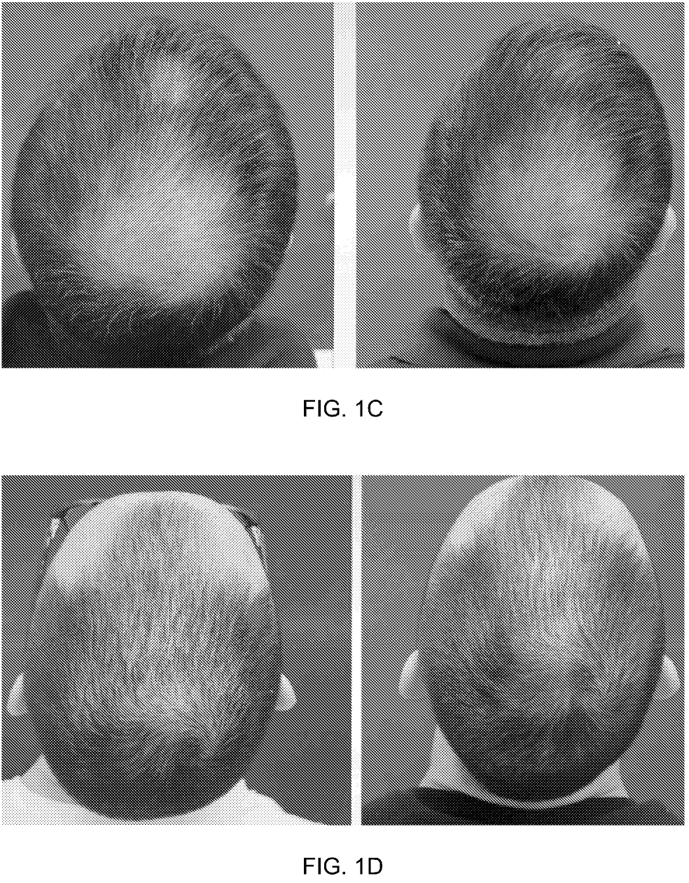

Five patients were selected for the purposes of this study. At three months, all five patients demonstrated increased hair density and new follicular growth. Improvement was noted in both the frontal zone and vertex areas. The area with the most apparent increased density was along the inferior border of the posterior crown area (see FIGS. 1A-1E). No side effects were reported during the initial study period, including a lack of sexual dysfunction or erectile dysfunction associated with oral dutasteride.

### Conclusion

The initial data demonstrates that the 2% w/w dutasteride topical liposomal gel was highly effective in halting the progression of pattern baldness. Also, the initial data demonstrate marked regrowth of affected follicles. Surprisingly, no local or systemic adverse effects were identified as a result of the 2% w/w dutasteride topical liposomal composition.

### Example 11: Clinical Topical Dutasteride Study

A clinical study was performed to assess the efficacy of weekly administration of the 2% w/w dutasteride formulation described in Example 5. Patients in the study were positively identified as having androgenic alopecia (male pattern baldness). The degree of baldness was subsequently assessed according to the Norwood Hamilton Scale. Individuals with more advanced loss on this scale were selected, as changes in hair loss density and new hair growth would be more easily assessed in these cases. Exclusion criteria included the use of dutasteride or any other hair loss treatment and/or hormonal therapy within the prior six months.

Patients were instructed to apply the 2% w/w dutasteride topical liposomal topical gel to the areas of the scalp affected by pattern baldness massaging the gel into the scalp. The patients were instructed to apply the gel once weekly after a morning shower, after having towel-dried the hair. Patients were instructed to not use hair-styling products during the course of the study.

All patients were informed of the known side-effects of oral dutasteride. They were instructed to contact the physician conducting the study should they experience any of these side-effects. In addition, they were instructed to report any other symptoms experienced, including local issues at the site of gel application.

Follow-up assessments conducted after 2 months of therapy. These assessments were documented using global photography of the scalp, focusing on areas affected by pattern baldness.

### Results

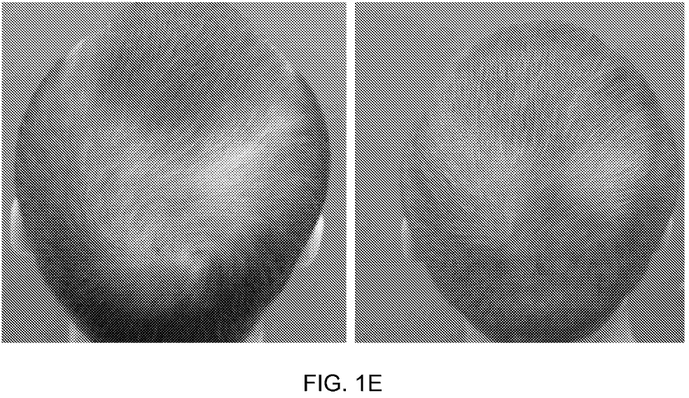

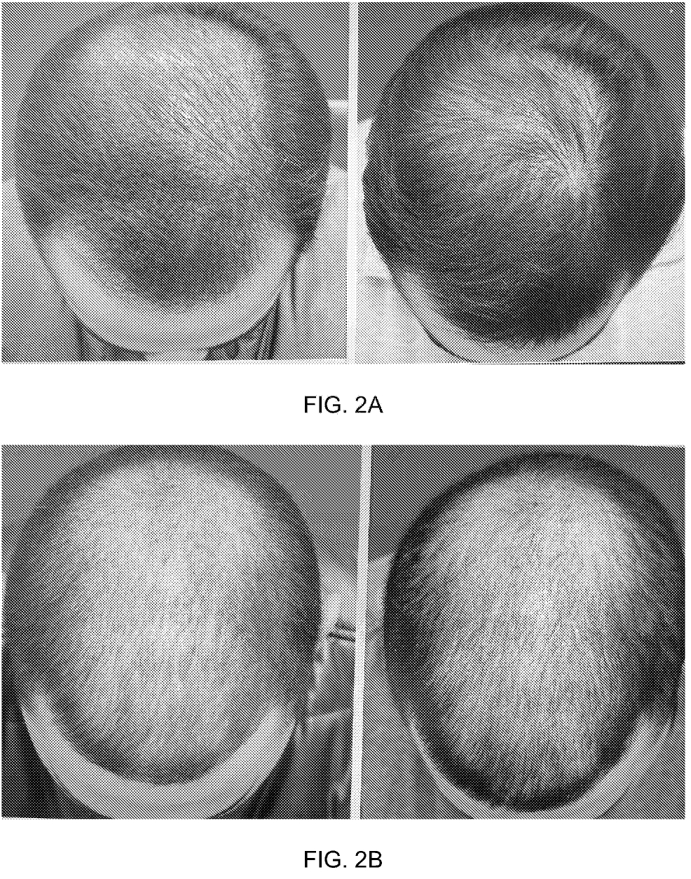

Two patients participated in this study. After two months of therapy, both patients demonstrated significantly increased hair density and new follicular growth. Improvement was noted in both the frontal zone and vertex areas. The area with the most apparent increased density was along the inferior border of the posterior crown area (see FIGS. 2A and 2B). No side effects were reported during the initial study period; specifically, the patients did not report any sexual dysfunction or erectile dysfunction, which are common side effect of oral dutasteride.

### Conclusion

The initial data demonstrate that weekly administration of the 2% w/w dutasteride topical liposomal gel was highly effective in halting the progression of pattern baldness. Also, the initial data demonstrate marked regrowth of affected follicles, reversal of follicular miniaturization, and reduction in the size of balding area. Surprisingly, no local or systemic adverse effects were identified as a result of the 2% w/w dutasteride topical liposomal composition.

All publications, patents, patent applications and other documents cited in this application are hereby incorporated by reference, including U.S. Provisional Appl. No. 63/019,335, in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the claimed invention(s).

What is claimed is:

1. A liposomal topical formulation, comprising:
a plurality of liposomes;
an aqueous gel matrix in which the plurality of liposomes are dispersed, wherein the aqueous gel matrix comprises a gelling agent, a water-soluble silicone compound, a film forming agent, and water; and
dutasteride entrapped within the liposomes.

2. The liposomal topical formulation of claim 1, wherein the film forming agent is a polysilsesquioxane compound.

3. The liposomal topical formulation of claim 2, wherein the polysilsesquioxane compound is a poly(alkylsilsesquioxane).

4. The liposomal topical formulation of claim 3, wherein the poly(alkylsilsesquioxane) is poly(methylsilsesquioxane).

5. The liposomal topical formulation of any one of the preceding claims, wherein the plurality of liposomes comprises lecithin.

6. The liposomal topical formulation of claim 5, wherein the lecithin is a hydrogenated lecithin.

7. The liposomal topical formulation of any one of the preceding claims, wherein the gelling agent is an acrylate polymer.

8. The liposomal topical formulation of claim 7, wherein the acrylate polymer is a C₁₀-C₃₀ alkyl acrylate crosspolymer.

9. The liposomal topical formulation of any one of the preceding claims, wherein the water-soluble silicone compound is a PEG-X silicone, wherein X is an integer of 6-20.

10. The liposomal topical formulation of claim 9, wherein the PEG-X silicone is PEG-8 dimethicone.

11. The liposomal topical formulation of any one of claims 1-8, wherein the water-soluble silicone compound is a PPG-X silicone, wherein X is an integer of 8-20.

12. The liposomal topical formulation of any one of the preceding claims, further comprising dimethyl isosorbide.

13. The liposomal topical formulation of any one of the preceding claims, further comprising an alcoholic solvent.

14. The liposomal topical formulation of claim 13, wherein the alcoholic solvent is propanediol, phenoxyethanol, aminomethyl propanol, or a combination thereof.

15. The liposomal topical formulation of any one of the preceding claims, further comprising a humectant.

16. The liposomal topical formulation of claim 14, wherein the humectant is caprylyl glycol, ethylhexylglycerin, or a mixture thereof.

17. The liposomal topical formulation of any one of the preceding claims, wherein the plurality of liposomes are at a concentration ranging from 0.1 to 1.0% by weight of the total composition.

18. The liposomal topical formulation of any one of the preceding claims, wherein the gelling agent has a concentration ranging from 0.3 to 1.3% by weight of the total composition.

19. The liposomal topical formulation of any one of the preceding claims, wherein the water-soluble silicone has a concentration ranging from 6 to 14 % by weight of the total composition.

20. The liposomal topical formulation of any one of claims 2-19, wherein the polysilsesquioxane compound has a concentration of 1-20% by weight of the total composition.

21. The liposomal topical formulation of claim 20, wherein the polysilsesquioxane compound has a concentration of 1-10% by weight of the total composition.

22. The liposomal topical formulation of claim 20, wherein the polysilsesquioxane compound has a concentration of 5-10% by weight of the total composition.

23. The liposomal topical formulation of claim 20, wherein the polysilsesquioxane compound has a concentration of 1-5% by weight of the total composition.

24. The liposomal topical formulation of any one of claims 2-19, wherein the polysilsesquioxane compound has a concentration of 10-20% by weight of the total composition.

25. The liposomal topical formulation of any one of claims 2-19, wherein the polysilsesquioxane compound has a concentration of at least 1% by weight of the total composition.

26. The liposomal topical formulation of claim 25, wherein the polysilsesquioxane compound has a concentration of at least 2% by weight of the total composition.

27. The liposomal topical formulation of claim 26, wherein the polysilsesquioxane compound has a concentration of at least 3% by weight of the total composition.

28. The liposomal topical formulation of claim 27, wherein the polysilsesquioxane compound has a concentration of at least 5% by weight of the total composition.

29. The liposomal topical formulation of any one of the preceding claims, further comprising an antifungal, an antibiotic, an anti-hypertensive vasodilator, a steroid, or combinations thereof.

30. The liposomal topical formulation of any one of the preceding claims, wherein the formulation comprises 0.5-5% w/w dutasteride.

31. The liposomal topical formulation of claim 30, wherein the formulation comprises 5% w/w dutasteride.

32. The liposomal topical formulation of claim 30, wherein the formulation comprises 4% w/w dutasteride.

33. The liposomal topical formulation of claim 30, wherein the formulation comprises 1-3% w/w dutasteride.

34. The liposomal topical formulation of claim 33, wherein the formulation comprises 3% w/w dutasteride.

35. The liposomal topical formulation of claim 33, wherein the formulation comprises 2% w/w dutasteride.

36. The liposomal topical formulation of any one of the preceding claims, wherein at least 75% of the total dutasteride in the formulation is entrapped in the liposomes.

37. The liposomal topical formulation of claim 36, wherein at least 90% of the total dutasteride in the formulation is entrapped within the liposomes.

38. A method of making a liposomal formulation, comprising mixing a plurality of liposomes with a gelling agent, a water-soluble silicone compound, a film forming agent, and dutasteride, such that at least a portion of the dutasteride becomes entrapped within the liposomes.

39. A method of making a liposomal formulation, comprising:
forming a plurality of liposomes; and
mixing the liposomes with a gelling agent, a water-soluble silicone compound, a film forming agent, and dutasteride, such that at least a portion of the dutasteride becomes entrapped within the liposomes.

40. The method of claim 39, wherein the liposomes are formed by mixing vesicle-forming lipids with water while stirring.

41. A method of making a liposomal formulation, comprising:
mixing a liposomal formulation base that comprises a plurality of liposomes with dutasteride, such that at least a portion of the dutasteride becomes entrapped within the plurality of liposomes;
wherein the liposomal formulation base further comprises a gelling agent, a water-soluble silicone compound, and a film forming agent.

42. The method of any one of claims 38-41, wherein the film forming agent is a polysilsesquioxane compound.

43. The method of claim 42, wherein the polysilsesquioxane compound is a poly(alkylsilsesquioxane).

44. The method of claim 43, wherein the poly(alkylsilsesquioxane) is poly(methylsilsesquioxane).

45. The method of any one of claims 38-44, wherein the plurality of liposomes comprises lecithin.

46. The method of claim 45, wherein the lecithin is a hydrogenated lecithin.

47. The method of any one of claims 38-46, wherein the gelling agent is an acrylate polymer.

48. The method of claim 47, wherein the acrylate polymer is a C₁₀-C₃₀ alkyl acrylate crosspolymer.

49. The method of any one of claims 38-48, wherein the water-soluble silicone compound is PEG-X silicone, wherein X is an integer of 6-20.

50. The method of claim 49, wherein the PEG-X silicone is PEG-8 dimethicone.

51. The method of any one of claims 38-48, wherein the water-soluble silicone compound is a PPG-X silicone, wherein X is an integer of 8-20.

52. The method of any one of claims 38-51, wherein the plurality of liposomes are at a concentration ranging from 0.1 to 1.0% by weight of the total composition.

53. The method of any one of claims 38-52, wherein the gelling agent has a concentration ranging from 0.3 to 1.3% by weight of the total composition.

54. The method of any one of claims 38-53, wherein the water-soluble silicone has a concentration ranging from 6 to 14% by weight of the total composition.

55. The method of any one of claims 38-54, wherein the polysilsesquioxane compound has a concentration of at least 3% by weight of the total composition.

56. The method of claim 55, wherein the polysilsesquioxane compound has a concentration of at least 5% by weight of the total composition.

57. The method of any one of claims 38-56, wherein the made liposomal formulation comprises 0.5-5% w/w dutasteride.

58. The method of claim 57, wherein the made liposomal formulation comprises 1-3% w/w dutasteride.

59. The method of claim 58, wherein the made liposomal formulation comprises 2% w/w dutasteride.

60. The method of any one of claims 38-59, wherein at least 75% of the total dutasteride in the formulation becomes entrapped in the liposomes.

61. The method of claim 60, wherein at least 90% of the total dutasteride in the formulation becomes entrapped in the liposomes.

62. Use of the liposomal topical formulation according to any one of claims 1-37 in the treatment or prevention of a dermatological disorder.

63. The use of claim 62, wherein the dermatological disorder is androgenic alopecia (male pattern baldness).

64. The use of claim 62, wherein the dermatological disorder is a fungal infection, a bacterial infection, acne, eczema, psoriasis, rosacea, vitiligo, or inflammation.

65. The use of any one of claims 62-64, wherein the use comprises administering the liposomal topical formulation once a week.

66. The use of any one of claims 62-64, wherein the use comprises administering the liposomal topical formulation once every 10 days.

67. The use of any one of claims 62-64, wherein the use comprises administering the liposomal topical formulation once every two weeks.

68. The use of any one of claims 62-64, wherein the use comprises administering the liposomal topical formulation once every other day.

69. The use of any one of claims 62-64, wherein the use comprises administering the liposomal topical formulation once every three days, once every four days, once every five days, or once every six days.

70. The use of any one of claims 62-64, wherein the use comprises administering the liposomal topical formulation twice per week, three times per week, four times per week, five times per week, or six times per week.

71. A method for treating or preventing a dermatological disorder, the method comprising administering the liposomal topical formulation according to any one of claims 1-37 to a patient in need thereof.

72. The method of claim 71, wherein the dermatological disorder is androgenic alopecia (male pattern baldness).

73. The method of claim 71, wherein the dermatological disorder is a fungal infection, a bacterial infection, acne, eczema, psoriasis, rosacea, vitiligo, or inflammation.

74. The method of any one of claims 71-73, wherein the method comprises administering the liposomal topical formulation once a week.

75. The method of any one of claims 71-73, wherein the method comprises administering the liposomal topical formulation once every 10 days.

76. The method of any one of claims 71-73, wherein the method comprises administering the liposomal topical formulation once every two weeks.

77. The method of any one of claims 71-73, wherein the method comprises administering the liposomal topical formulation once every other day.

78. The method of any one of claims 71-73, wherein the method comprises administering the liposomal topical formulation once every three days, once every four days, once every five days, or once every six days.

79. The method of any one of claims 71-73, wherein the method comprises administering the liposomal topical formulation twice per week, three times per week, four times per week, five times per week, or six times per week.

80. A liposomal topical formulation, comprising:
a plurality of liposomes;
an aqueous gel matrix in which the plurality of liposomes are dispersed, wherein the aqueous gel matrix comprises a gelling agent, a water-soluble silicone compound, and water; and
dutasteride entrapped within the liposomes.

81. The liposomal topical formulation of claim 80, wherein the plurality of liposomes comprises lecithin.

82. The liposomal topical formulation of claim 81, wherein the lecithin is a hydrogenated lecithin.

83. The liposomal topical formulation of any one of claims 80-82, wherein the gelling agent is an acrylate polymer.

84. The liposomal topical formulation of claim 83, wherein the acrylate polymer is a C₁₀-C₃₀ alkyl acrylate crosspolymer.

85. The liposomal topical formulation of any one of claims 80-84, wherein the water-soluble silicone compound is PEG-X silicone, wherein X is an integer of 6-20.

86. The liposomal topical formulation of claim 85, wherein the water-soluble silicone compound is PEG-8 dimethicone.

87. The liposomal topical formulation of any one of claims 80-84, wherein the water-soluble silicone compound is a PPG-X silicone, wherein X is an integer of 8-20.

88. The liposomal topical formulation of any one of claims 80-87, further comprising dimethyl isosorbide.

89. The liposomal topical formulation of any one of claims 80-88, further comprising an alcoholic solvent.

90. The liposomal topical formulation of claim 89, wherein the alcoholic solvent is propanediol, phenoxyethanol, aminomethyl propanol, or a combination thereof.

91. The liposomal topical formulation of any one of claims 80-90, further comprising a humectant.

92. The liposomal topical formulation of claim 91, wherein the humectant is caprylyl glycol, ethylhexylglycerin, or a mixture thereof.

93. The liposomal topical formulation of any one of claims 80-92, wherein the plurality of liposomes are at a concentration ranging from 0.1 to 1.0% by weight of the total composition.

94. The liposomal topical formulation of any one of claims 80-93, wherein the gelling agent has a concentration ranging from 0.3 to 1.3% by weight of the total composition.

95. The liposomal topical formulation of any one of claims 80-94, wherein the water-soluble silicone has a concentration ranging from 6 to 14% by weight of the total composition.

96. The liposomal topical formulation of any one of claims 80-95, wherein the formulation comprises 0.5-5% w/w dutasteride.

97. The liposomal topical formulation of claim 96, wherein the formulation comprises 1-3% w/w dutasteride.

98. The liposomal topical formulation of claim 97, wherein the formulation comprises 2% w/w dutasteride.

99. The liposomal topical formulation of any one of claims 80-98, wherein at least 75% of the total dutasteride in the formulation is entrapped in the liposomes.

100. The liposomal topical formulation of claim 99, wherein at least 90% of the total dutasteride in the formulation is entrapped in the liposomes.

101. A method of making a liposomal formulation, comprising mixing a plurality of liposomes with a gelling agent, a water-soluble silicone compound, and dutasteride, such that at least a portion of the dutasteride becomes entrapped within the liposomes.

102. A method of making a liposomal formulation, comprising:
forming a plurality of liposomes; and
mixing the liposomes with a gelling agent, a water-soluble silicone compound, and dutasteride, such that at least a portion of the dutasteride becomes entrapped within the liposomes.

103. The method of claim 102, wherein the liposomes are formed by mixing vesicle-forming lipids with water while stirring.

104. A method of making a liposomal formulation, comprising:
mixing a liposomal formulation base that comprises a plurality of liposomes with dutasteride, such that at least a portion of the dutasteride becomes entrapped within the plurality of liposomes;
wherein the liposomal formulation base further comprises a gelling agent and a water-soluble silicone compound.

105. The method of any one of claims 101-104, wherein the plurality of liposomes comprises lecithin.

106. The method of claim 105, wherein the lecithin is a hydrogenated lecithin.

107. The method of any one of claims 101-106, wherein the gelling agent is an acrylate polymer.

108. The method of claim 107, wherein the acrylate polymer is a C₁₀-C₃₀ alkyl acrylate crosspolymer.

109. The method of any one of claims 101-108, wherein the water-soluble silicone compound is PEG-X silicone, wherein X is an integer of 6-20,.

110. The method of claim 109, wherein the PEG-X silicone is PEG-8 dimethicone.

111. The method of any one of claims 101-108, wherein the water-soluble silicone compound is a PPG-X silicone, wherein X is an integer of 8-20.

112. The method of any one of claims 101-111, wherein the plurality of liposomes are at a concentration ranging from 0.1 to 1.0% by weight of the total composition.

113. The method of any one of claims 101-112, wherein the gelling agent has a concentration ranging from 0.3 to 1.3% by weight of the total composition.

114. The method of any one of claims 101-113, wherein the water-soluble silicone has a concentration ranging from 6 to 14% by weight of the total composition.

115. The method of any one of claims 101-114, wherein the made liposomal formulation comprises 0.5-5% w/w dutasteride.

116. The method of claim 115, wherein the made liposomal formulation comprises 1-3% w/w dutasteride.

117. The method of claim 116, wherein the made liposomal formulation comprises 2% w/w dutasteride.

118. The method of any one of claims 101-117, wherein at least 75% of the total dutasteride in the formulation becomes entrapped in the liposomes.

119. The method of claim 118, wherein at least 90% of the total dutasteride in the formulation becomes entrapped in the liposomes.

120. Use of the liposomal topical formulation according to any one of claims 80-100 in the treatment or prevention of androgenic alopecia (male pattern baldness).

121. A method for treating androgenic alopecia (male pattern baldness), the method comprising administering a topical formulation comprising dutasteride to a patient in need thereof.

122. The method of claim 121, wherein the topical formulation comprises 1-3% w/w dutasteride.

123. The method of claim 122, wherein the topical formulation comprises 2% w/w dutasteride.

124. The method according to any one of claims 121-123, wherein the method comprises administering the topical formulation daily.

125. The method according to any one of claims 121-123, wherein the method comprises administering the topical formulation once a week.
